(19) United States
(12) Patent Application Publication
(10) Pub. No.: US 2026/0210937 A1
(43) Pub. Date: Jul. 23, 2026

(54) APPARATUS AND METHOD FOR MEASURING WORKABILITY OF CONCRETE

(71) Applicant: BOARD OF REGENTS FOR THE OKLAHOMA AGRICULTURAL AND MECHANICAL COLLEGES, STILLWATER, OK (US)

(72) Inventors: Matthew Tyler Ley, Stillwater, OK (US); Mohammad Jobaer Uddin, Stillwater, OK (US); Marllon Daniel Cook, Stillwater, OK (US)

(21) Appl. No.: 19/138,369

(22) PCT Filed: Dec. 12, 2023
(86) PCT No.: PCT/US2023/083587

§ 371 Date: Jun. 12, 2025

Related U.S. Application Data
(60) Provisional application No. 63/431,885, filed on Dec. 12, 2022.
(60) Provisional application No. 63/439,289, filed on Jan. 17, 2023.

(51) Int. Cl. G01N 33/38; G01N 11/00; G01N 11/02

(52) U.S. Cl. CPC G01N 33/383; G01N 11/02; G01N 2011/0053; G01N 2011/0073

(57) ABSTRACT

Apparatus and method for characterizing concrete use measurements of vibration damping in the concrete to determine workability and describe the consistency of different batches of concrete as well as the behavior of concrete during consolidation. Accelerometers are used to measure vibration transmitted from a source of vibration within a sample to the outer surface of a container holding the sample. Curves of acceleration versus time measured by the accelerometers are correlated with the fresh property characteristics of the concrete.

### CROSS REFERENCE TO RELATED APPLICATIONS

This application is based upon and claims priority to US Provisional Application No. U.S. 63/431,885, filed on Dec. 12, 2022, and U.S. Provisional Application No. 63/439,289, filed on Jan. 17, 2023. Both applications are hereby incorporated by reference herein in their entireties.

### FIELD

The disclosure concerns an apparatus and method for measuring characteristics of concrete, particularly the workability of slip form concrete.

### BACKGROUND

Laying concrete using a slip form paver requires experienced and skillful operators to run the paver at the proper travel speed with the proper vibration rate and auger rate to ensure the formation of concrete that is well consolidated, smooth, and does not edge slump or decrease in height significantly when it leaves the paver. It is thought that there is too much reliance on the skill and judgement of the operator, resulting in inferior slabs laid by less experienced operators who cannot properly judge the required slip paver operating parameters for a particular batch of concrete. There is clearly an opportunity to improve slip form paver operation by reducing reliance on operator skill using real-time and on-site measurements of concrete characteristics which affect slip paver operation.

### BRIEF DESCRIPTION OF THE DRAWINGS

FIG. 1 illustrates an example apparatus for measuring workability of concrete according to the disclosure and according to an example method according to the disclosure;

FIG. 2 illustrates a component used in the apparatus shown in FIG. 1;

FIGS. 3 and 3A illustrate components used in the apparatus shown in FIG. 1;

FIGS. 4 and 5 illustrate correlation between the workability of concrete and measurements made using the apparatus and method according to the disclosure;

FIGS. 6 and 7 illustrate correlation between the workability of concrete and measurements made using the apparatus and method according to the disclosure;

FIG. 8 is a graph summarizing the correlation between concrete workability and measurements made using the apparatus and method according to the disclosure;

FIGS. 9 and 10 illustrate the use of the apparatus to determine consistency between different mixes of concrete;

FIG. 11 illustrates the use of the apparatus and method according to the disclosure to characterize concrete during consolidation;

FIG. 12 illustrates the effect of fine sand content variation on vibration response in fresh concrete;

FIG. 13 illustrates example acceleration data collected using the apparatus and method according to the disclosure;

FIGS. 14 and 15 illustrate examples of preprocessing data collected using the apparatus and method according to the disclosure;

FIGS. 16-18 illustrates further aspects, specifically velocity, displacement, g-factor, spectrogram, and Fast Fourier Transformation, which can be determined from the data collected using the apparatus and method according to the disclosure to further analyze the data;

FIGS. 20-29 illustrate the correlation between the workability of concrete, data collected, and calculations made using the apparatus and method according to the disclosure;

FIG. 30 illustrates an example apparatus for measuring the thickness of mortar at the surface of fresh consolidated concrete according to the disclosure and according to an example method according to the disclosure;

FIGS. 31 and 32 illustrate the method of using the apparatus of FIG. 30 to measure the thickness of mortar; and

FIG. 33 illustrates the correlation between the mortar volume and the data collected using the apparatus of FIG. 30 and method according to the disclosure.

### DETAILED DESCRIPTION

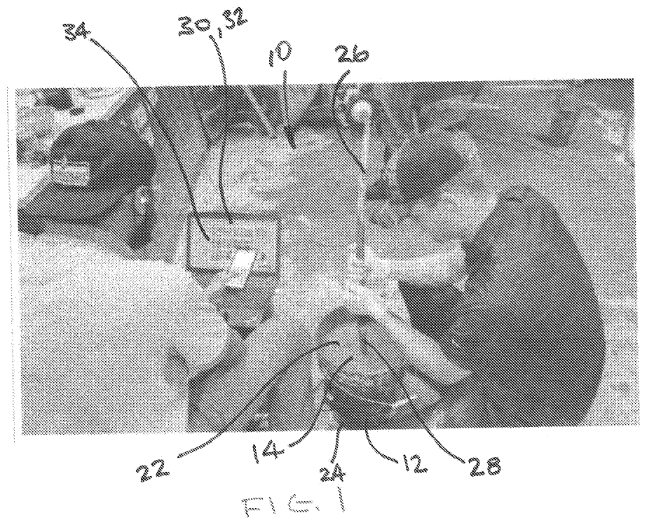

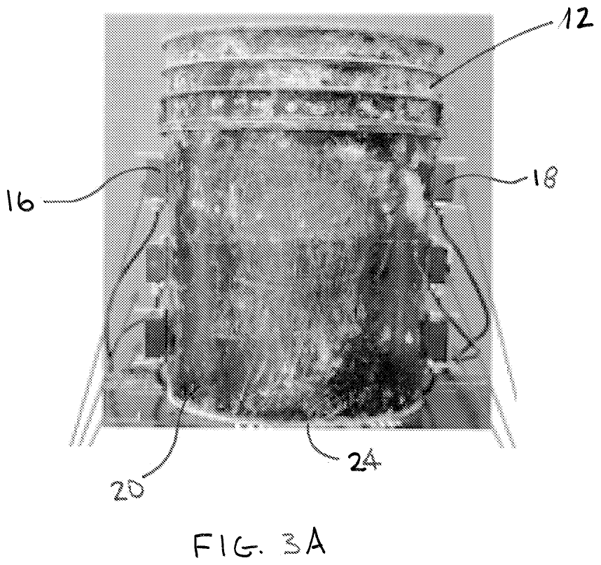

FIG. 1 shows an apparatus 10 for measuring the workability of concrete. In this example the apparatus 10 comprises a container 12 adapted to receive a sample 14 of the concrete. FIG. 3 shows an example container 12 in detail, the container comprising a cylindrical bucket formed of plastic resin and having an internal volume of approximately five gallons. First and second vibration sensors 16 and 18 are mounted diametrically opposite to one another on an outer surface 20 of the container 12. In a practical example embodiment the sensors 16 and 18 may comprise accelerometers having a sampling rate of 200 Hz, a baud rate of 115,200 bits per second and a band width of 256 Hz. Sensors with a sampling rate of 400 Hz are also advantageous as this is twice the frequency of typical vibrators used in concrete. This would allow greater insight into observations of the frequency domain of the concrete. However, accelerometers with other sample rates can be used if at least a component of the signal is captured by the accelerometer. The first and second vibration sensors 16 and 18 may have the same sampling rate. Optionally, the first sensor 16 may have a different sampling rate than the second sensor 18. For example, the first sensor 16 may have a sampling rate of 200 Hz and the second sensor 18 may have a sampling rate of 400 Hz. The accelerometers 16 and 18 are adapted to measure the magnitude of acceleration at the outer surface 20 of the container 12 and generate signals indicative of this magnitude as a function of time. Although it is thought feasible to use only one accelerometer, it is considered advantageous to use two accelerometers 16 and 18 mounted on opposite sides of the container 12 as shown. The accelerometers are furthermore positioned on the container 12 to measure the magnitude of vibration at a point on the container approximately half way between a free surface 22 of the sample (see FIG. 1) and the bottom 24 of the container 12 (see also FIG. 3). In this example, the first and second accelerometers 16 and 18 are bolted to the outer surface 20 of the container, other mounting arrangements and increased number of accelerometers at different heights being of course feasible. For example, as shown in FIG. 3A, two groups of three sensors 16 and 18 are positioned on the outer surface 20 of the container 12. The sensors are positioned at different heights above the bottom 24 of the container 12.

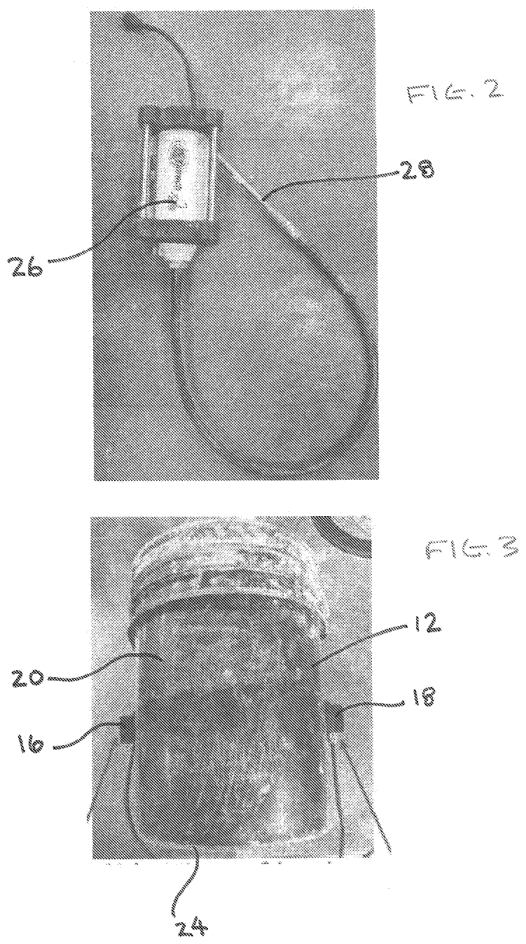

FIG. 1 also shows a vibrator 26 adapted to be positioned in the sample 14 within the container 12. In a practical example embodiment, vibrator 26 may comprise a control speed electric flexible shaft concrete vibrator as manufactured by Minnich Manufacturing, Inc. As shown in FIG. 2, the vibrator 26 has a 0.75″ vibratory head 28 capable of vibrating at 10,500 cycles per minute and producing an acceleration at the head of about 20 gs for sustained periods of time. Other vibrating frequencies and head sizes may also be advantageous for certain mixtures to better determine their properties. It is also possible to use different makes and models that have a frequency that can be measured.

As shown in FIG. 1 the vibratory head 28 of the vibrator 26 is adapted to be positioned within the geometric center of the sample 14 and apply vibrations within the sample. It is thought advantageous to position the vibratory head 28 within the geometric center of the sample so the signals from the accelerometers 16 and 18 are not distorted by reflections within the container 12 or by the head being closer to one sensor than the other. Also, this ensures that the travel path of the vibration energy through the concrete is similar for both accelerometers. It is further considered advantageous to preposition the vibrator 26 within the container before the sample is placed within the container. Prepositioning the vibrator provides a better understanding of the signals because if the vibrator is inserted into the fresh concrete while vibrating this means the speed of travel of the vibrator has to be accounted for. Prepositioning the vibrator may help capture the first seconds of energy transfer until final consolidation and may ensure that the energy will be transferred to the accelerometers without moving the vibrator thereby making it easier to understand the measurements recorded by the device.

When the vibrator 26 is activated within the concrete sample 14 the accelerometers 16 and 18 measure the magnitude and frequency of vibration generated by the head and transmitted through the sample to the outer surface 20 of the container 12. The accelerometers 16 and 18 generate signals indicative of the magnitude of the vibration, the signals being an indication of the damping or energy transfer provided by the concrete before hardening/in the plastic state.

FIG. 1 also shows a data collection and display device 30 for receiving the signals from the first and second accelerometers. In this example the device 30 comprises a laptop computer 32 having a visual display, screen 34, for displaying the magnitude of vibration measured by the sensors 16 and 18 as a function of time. Device 30 may further have one or more electronic filters for conditioning the signals as well as resident software to collect and store data, perform various mathematical analyses and drive the display. Other ways to collect and display the data are also possible.

The disclosure further encompasses a method of measuring workability of concrete. Other words for workability may include slump, flowability, response to vibration, and energy required to displace or move the concrete. An example embodiment of a method according to the disclosure is shown in FIG. 1 and comprises:
adding concrete within a container 12;
positioning a vibratory head 28 into the container;
applying vibration within the sample 14 by activating the vibrator 26;
measuring a magnitude of vibration transmitted through the sample to the container 12 (accelerometers 16 and 18 measure acceleration at the surface of container 12 and generate signals indicative of the magnitude of the vibrations and transmit the signals to the data collection and display device 30);
evaluating the magnitude and frequency of vibration to determine the workability of the concrete (computer 34 evaluates and displays the measurements).

As shown in FIG. 1, the vibratory head 28 is inserted into the geometric center of the sample 14. The accelerometers 16, 18 measure the magnitude of the vibration at points on the container opposite to one another (see also FIG. 3). The accelerometers 16, 18 are also positioned to measure the vibration at a point on the container approximately half way between a free surface 12 of the sample and a bottom 24 of the container 12. The signals from the accelerometers are indicative of the magnitude and frequency of the vibration of the concrete between the vibrator and the accelerometer and in turn the acceleration of the walls of the container as they are in contact with the concrete. These results are displayed as a function of time on data collection and display device 30.

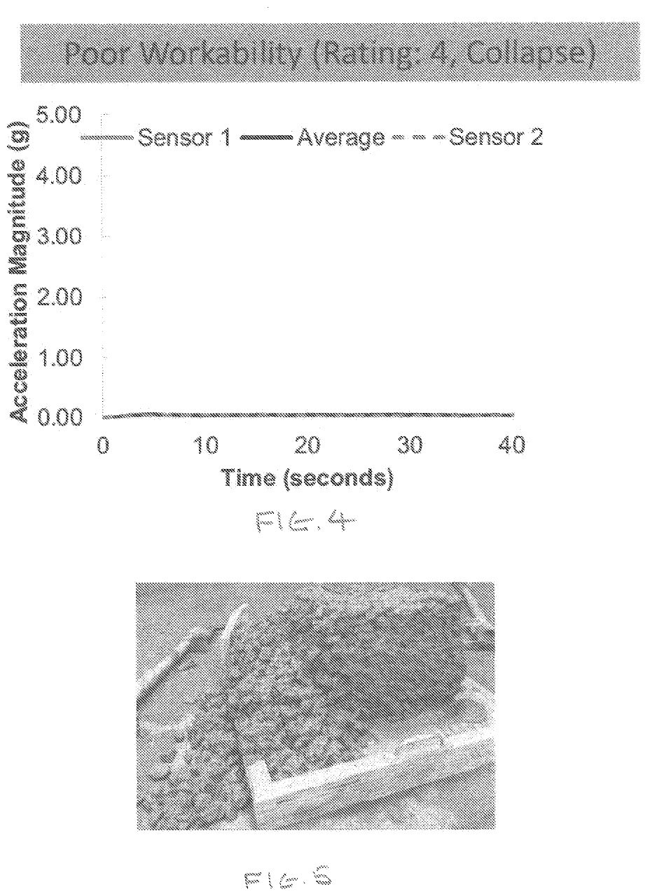

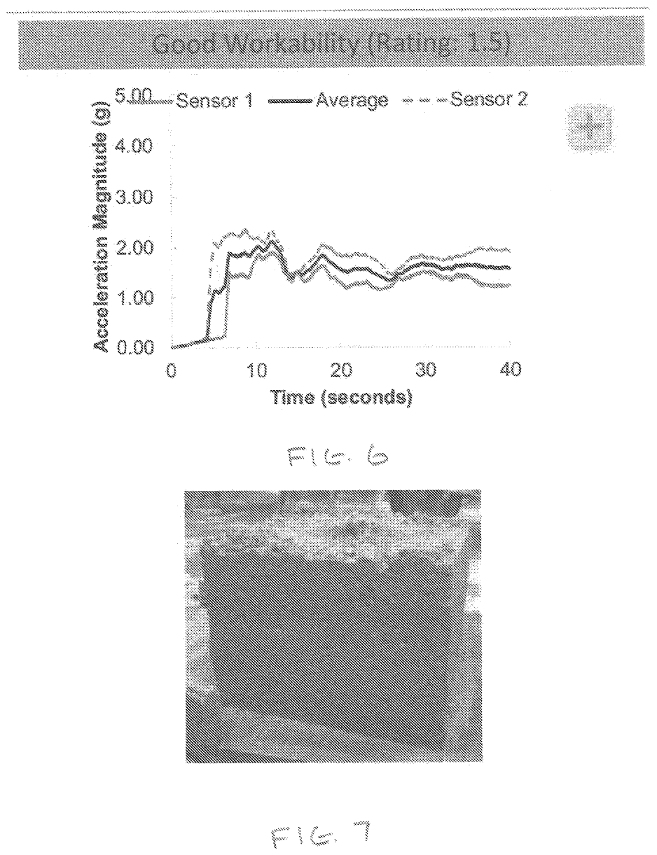

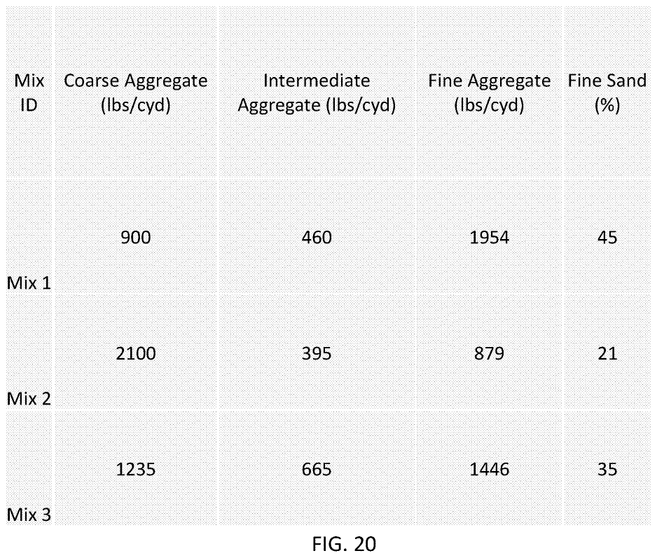

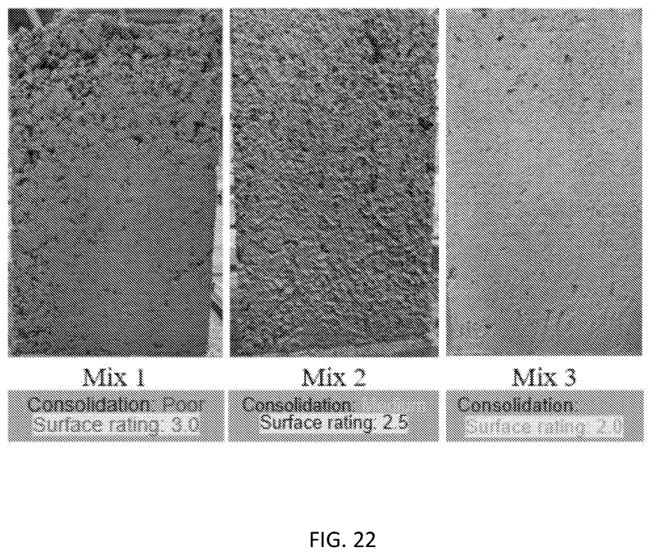

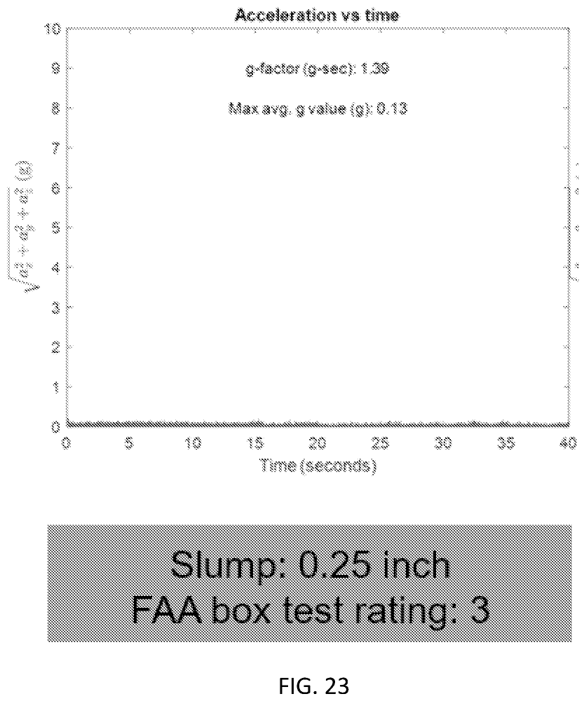

FIGS. 4-7 illustrate the correlation between the vibration magnitude, as measured using an apparatus and method according to the disclosure, and the workability of the concrete. FIG. 4 is a plot showing vibration test results conducted on a first batch of concrete. The vibration magnitudes measured by the accelerometers along with the averaged signal, are plotted on the Y axis, against time plotted on the x axis. The plot in FIG. 4 shows that essentially no vibration is transmitted through the sample indicating high vibration damping. FIG. 5 shows the results of a box test according to AASTHO T 396-22 performed on the same batch of concrete. The box test results in a rating of 4, indicating poor workability. Thus high damping, i.e., poor vibration transmission through the concrete, correlates with poor workability and performance in the box test. As shown in FIG. 5, the sample from the first batch of concrete has collapsed upon box removal. Similar results are shown in FIG. 23. Similar to FIG. 4, FIG. 23 shows the plot of vibration magnitudes measured by accelerometers having a higher sampling rate plotted on the Y axis, against time plotted on the x axis. The plot in FIG. 23 shows that essentially no vibration is transmitted through Mix 1 shown in FIG. 20 indicating high vibration damping. As shown in FIG. 22, the results of a box test performed on Mix 1 indicated a surface rating of 3.

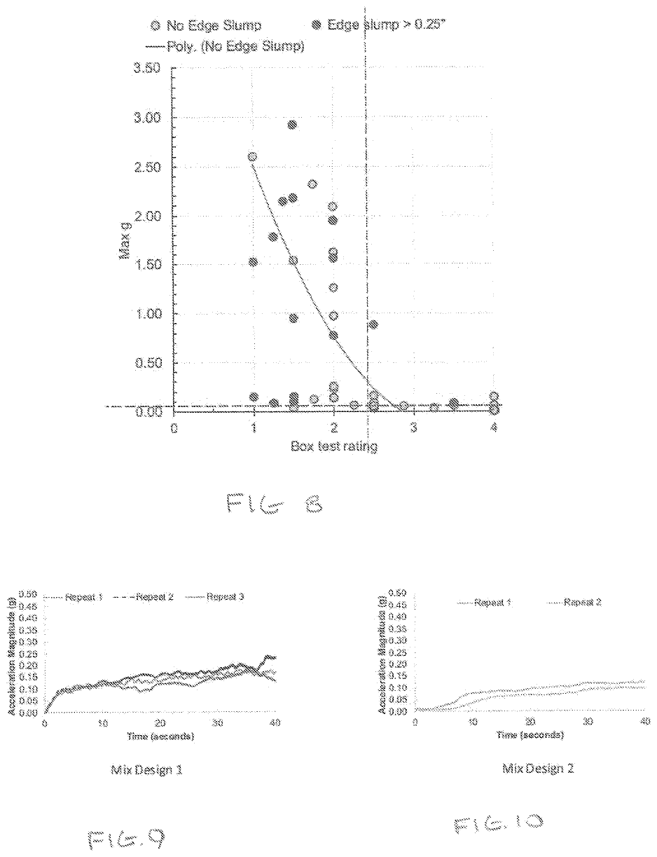

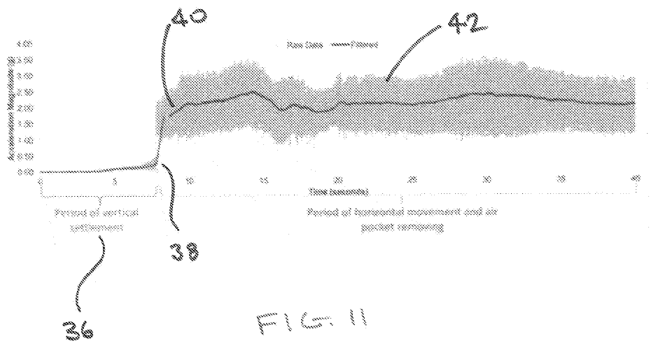

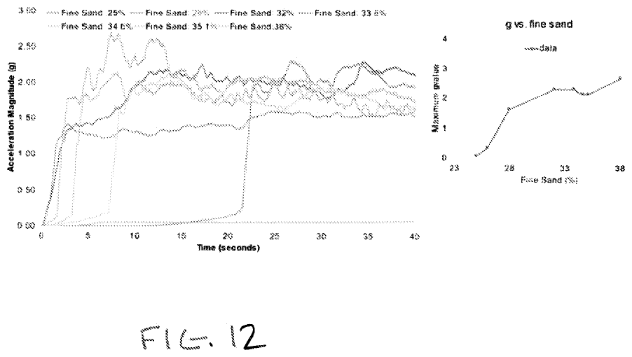

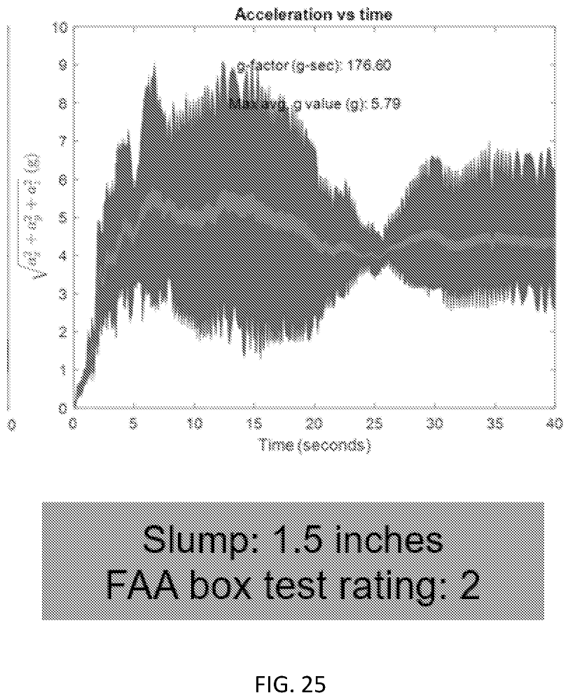

FIG. 6 is a plot showing the results of a vibration test conducted on a second batch of concrete using an apparatus and method according to the disclosure. In contrast to FIG. 5, the plot of FIG. 6 shows strong signals from the accelerometers, indicating excellent transmission of vibration through the second concrete batch (about 2.33 g measured at the surface of the container from a 20 g magnitude disturbance at the center of the sample, a transmission rate of almost 12%). FIG. 7 shows the results of a box test conducted on the second batch of concrete, indicating a rating of 1.5, good workability. Thus lower damping, i.e., good vibration transmission through the concrete, correlates with good workability. These results are further illustrated in FIG. 8, which plots vibration magnitude on the Y axis and box test rating on the X axis. Similar results are shown in FIG. 25. Similar to FIG. 6,

FIG. 25 shows the plot of vibration magnitudes measured by accelerometers having a higher sampling rate plotted on the Y axis, against time plotted on the x axis. The plot in FIG. 25 shows strong signals from the accelerometers, indicating excellent transmission of vibration through Mix 3 shown in FIG. 20. As shown in FIG. 22, the results of a box test performed on Mix 3 indicated a surface rating of 2.

The apparatus according to the disclosure may also be used in a method for comparing a consistency between a first batch of concrete and a second batch of concrete, the method also being encompassed by the disclosure. In an example embodiment, the method comprises:
positioning a first sample of the first batch within a container;
positioning a vibratory head 28 into the container;
applying vibration within the first sample;
measuring a magnitude of vibration transmitted through the first sample to the container;
removing the first sample from the container;
positioning a second sample of the second batch within the container;
positioning a vibratory head 28 into the container;
applying vibration within the second sample;
measuring a magnitude of vibration transmitted through the second sample to the container;
comparing the magnitude of vibration transmitted through the first sample to the container with the magnitude of vibration transmitted through the second sample to the container.

The results of such a test are shown by a comparison of FIGS. 9 and 10, which show the signals generated by the accelerometers as a function of time for two different concrete mixes. Mix design 1, (results shown in FIG. 9) comprises 24.1% by volume paste; w/c of 0.45; water reducer of 9 oz/hundred weight of cementitious and 28% fine sand. The combined gradation of the coarse and fine aggregates also meet the limits of the Tarantula Curve. Mix design 2 (results shown in FIG. 10) comprises a repeat of the same mixture. The comparison of FIGS. 9 and 10 show that the results are repeatable. This shows that the vibration magnitude is sensitive to changes in the specific mix design, and it is expected that the method and apparatus according to the disclosure may be used to determine consistency between batches of concrete and give insight to changes in the concrete mixtures.

Vibration responses and amount of surface voids can be influenced by the aggregate gradations (amount and size distribution of coarse, intermediate and fine aggregate). Based on the measurements, the device can detect changes in the aggregate gradation that cause changes in the vibrational energy transfer. FIG. 8 shows how different aggregate gradations impacts the measured energy transfer. FIG. 12 shows how the fine sand content impacts the energy transferred. This gives users a useful tool to help design their concrete mixture for the desired energy transfer and to understand how changes in the fine sand content can change their results. Note, fine sand content is the summation of aggregate retained on the ≤#30 sieve.

Research indicates that the apparatus and method according to the disclosure may be used to characterize further concrete behavior related to consolidation and water separation. FIG. 11 shows accelerometer test data correlated with a period of vertical settlement of the concrete 36 (essentially no vibration transmission through the concrete as the energy is used to rearrange the structure of the aggregates), the initiation of localized bleeding or water at the surface 38, marked by a steep rise in the vibration transmission, followed by an indication of global bleeding by a change in the slope 40, indicating a reduction in the rate of change of vibration transmission and an eventual flattening of the curve 42, possibly indicating water separation, horizontal movement of the concrete and air pocket removal.

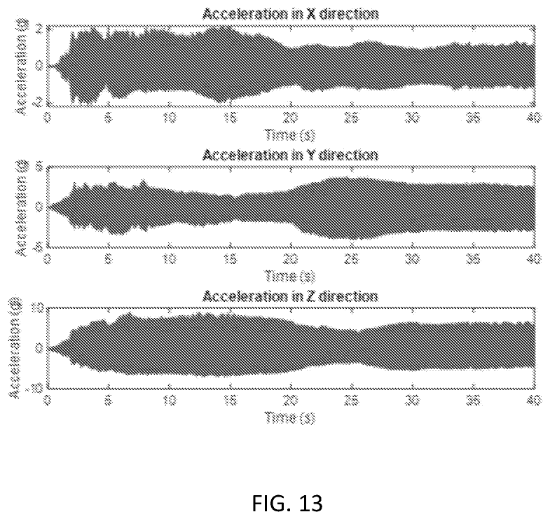

FIGS. 13-19 illustrate a correlation between the vibration magnitude, as measured using an apparatus and method according to the disclosure, and the workability of the concrete. As shown in FIG. 13, the acceleration from the first and/or second accelerometers is recorded in three directions, X, Y, Z.

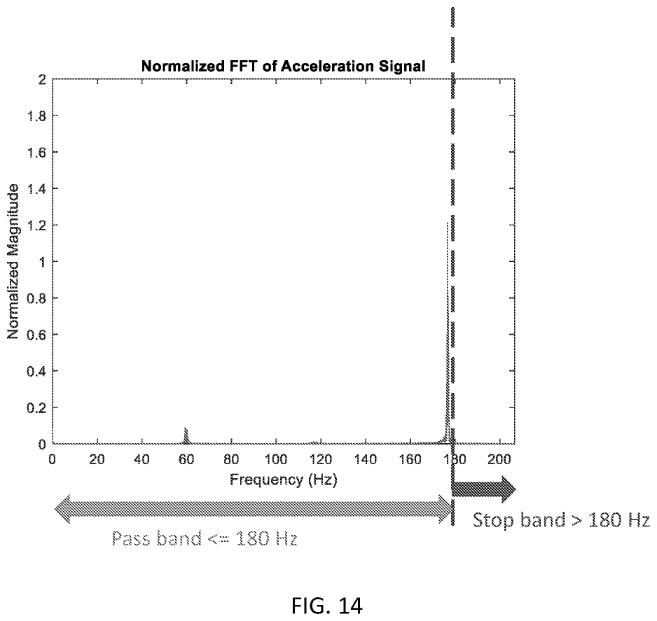

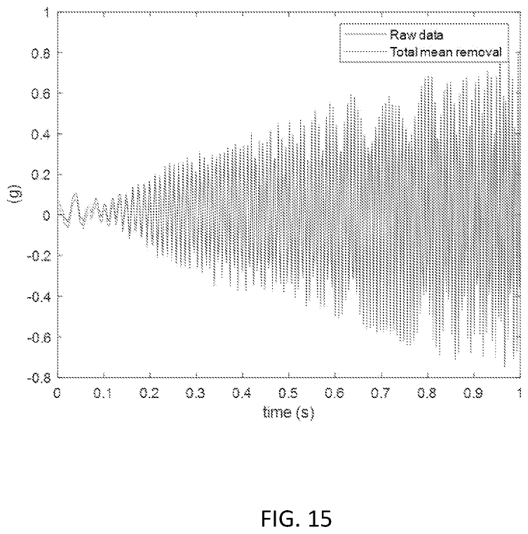

As shown in FIG. 14, the acceleration data may be preprocessed by applying a low pass filter in which unwanted high frequency noise is removed from the data. Optionally, a frequency greater than 180 Hz may be removed. As shown in FIG. 15, the acceleration date may be preprocessed by applying a total mean removal to remove any unwanted drift from the accelerometer data.

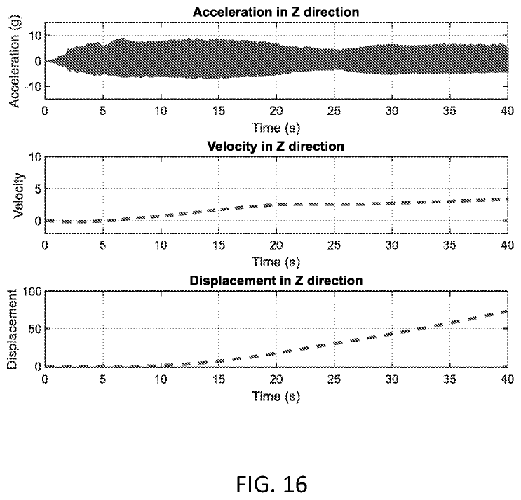

As shown in FIG. 16, the time domain velocity and displacement analysis may be calculated by integrating the acceleration signals along the three different directions, X, Y, Z (the Z-axis is shown as an example). The velocity is obtained by integrating the acceleration, and the displacement is obtained by further integrating the velocity. This process may be applied to the acceleration data from each axis to obtain the corresponding velocity and displacement data. The time domain velocity and displacement analysis may be used to provide further insight and analysis of the data.

The accelerometer data may be used to determine magnitude value by taking the square root of the acceleration in the X direction, squared, plus the acceleration in the Y direction, squared, plus the acceleration in the Z direction, squared, as shown below:

$$\vert M \vert = \sqrt{( {a}_{x}^{2} + {a}_{y}^{2} + {a}_{z}^{2} )}$$

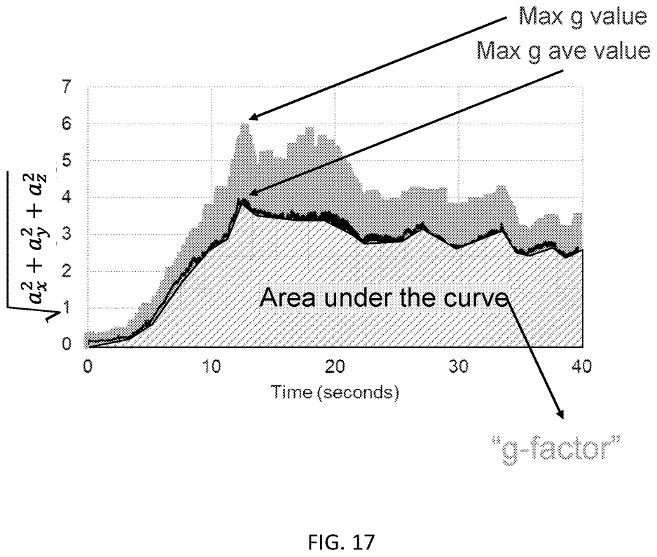

As shown in FIG. 17, the magnitude, or “g” value, may be plotted over time. The magnitude value may undergo transformations over time as the rheological properties of the concrete change due to change in mix design parameters. As shown in FIG. 17, the average magnitude may also be plotted over time. The area under this curve may be calculated to determine a “g-factor.”

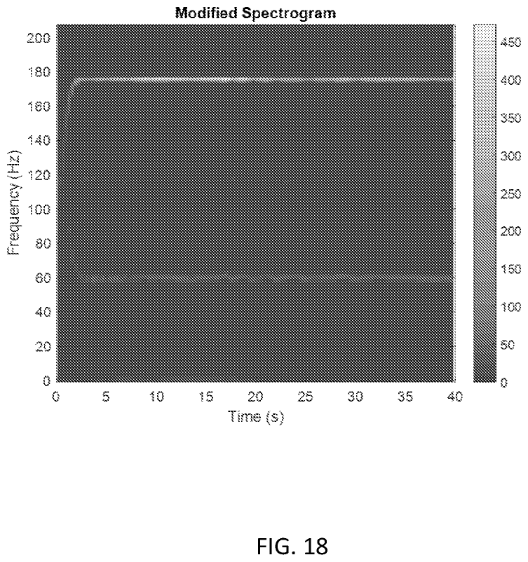

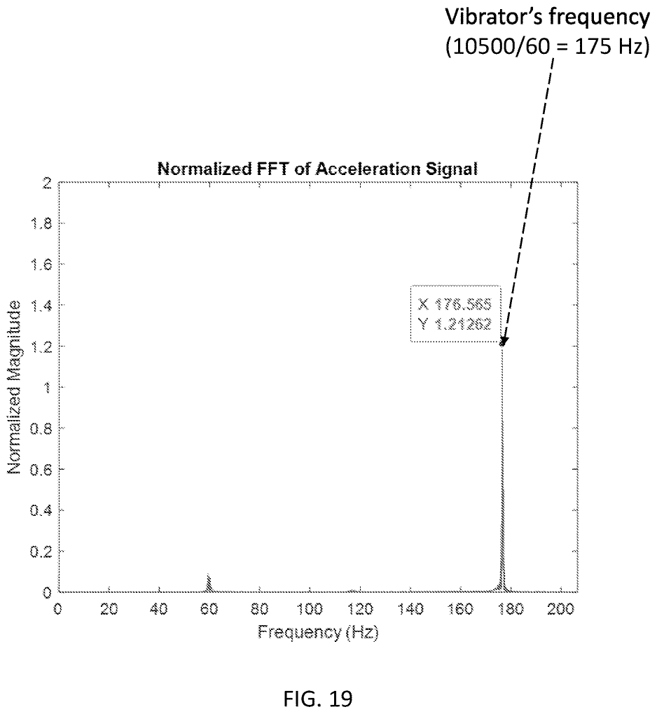

As shown in FIG. 18, a spectrogram may show how the signal and amplitude evolves over time. As shown in FIG. 19, a Fast Fourier Transformation (FFT) plot of the signal may indicate the strongest amplitude at vibrating frequency. Usage of spectrogram and FFT may be useful to investigate the data from a frequency domain perspective which may help identify patterns, anomalies, or specific conditions based on the frequency content of the vibrations. The spectrogram plots may allow for the identification of changes in frequency and magnitude over time, which are important aspects of a dynamic system.

The apparatus according to the disclosure may also be used in a method for comparing a consistency between concrete mixtures, the method also being encompassed by the disclosure. In an example embodiment, the method comprises:
positioning a first sample of the first batch within a container;
positioning a vibratory head into the container;
applying vibration within the first sample;
measuring a magnitude of vibration transmitted through the first sample to the container;
plotting the magnitude of vibration transmitted through the first sample to the container over time;
calculating the area under the curve in the plot to determine a g-factor;
removing the first sample from the container;
positioning a second sample of the second batch within the container;
positioning a vibratory head 28 into the container;
applying vibration within the second sample;
measuring a magnitude of vibration transmitted through the second sample to the container;
plotting the magnitude of vibration transmitted through the second sample to the container over time;
calculating the area under the curve in the plot to determine a g-factor;
comparing the g-factor resulting from the magnitude of vibration transmitted through the first sample to the container with the g-factor resulting from the magnitude of vibration transmitted through the second sample to the container.

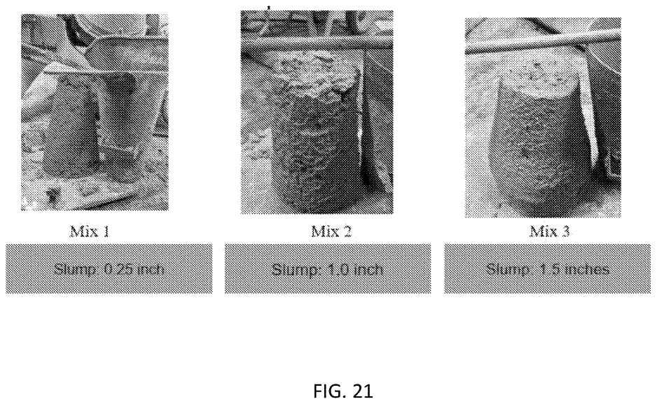

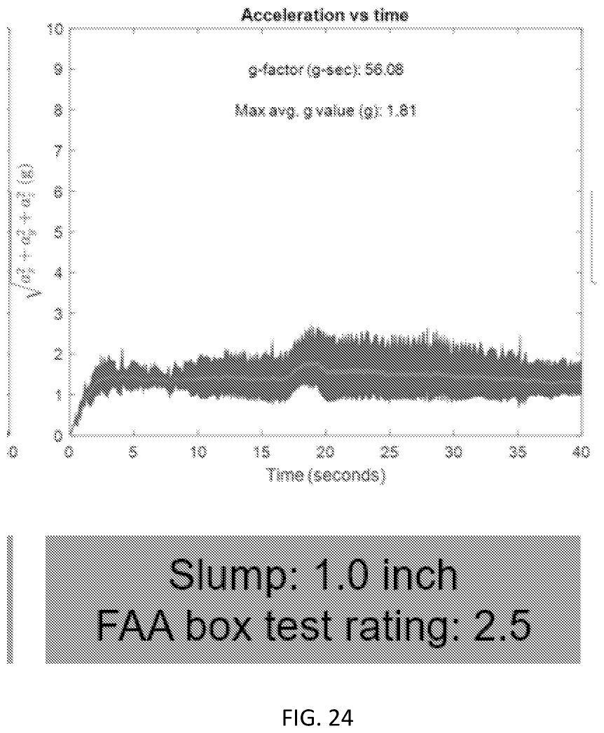

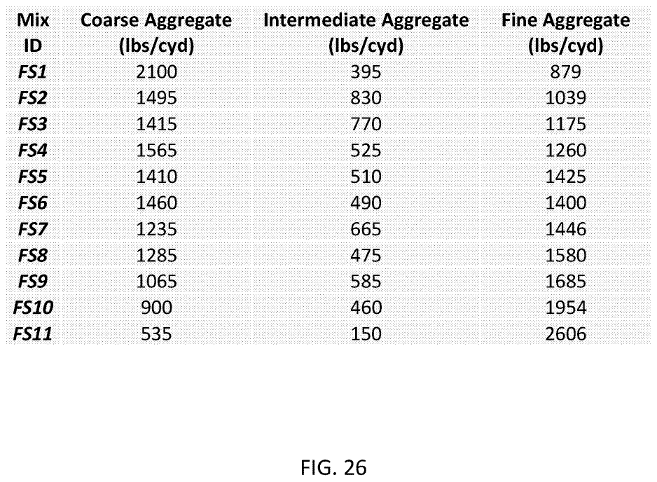

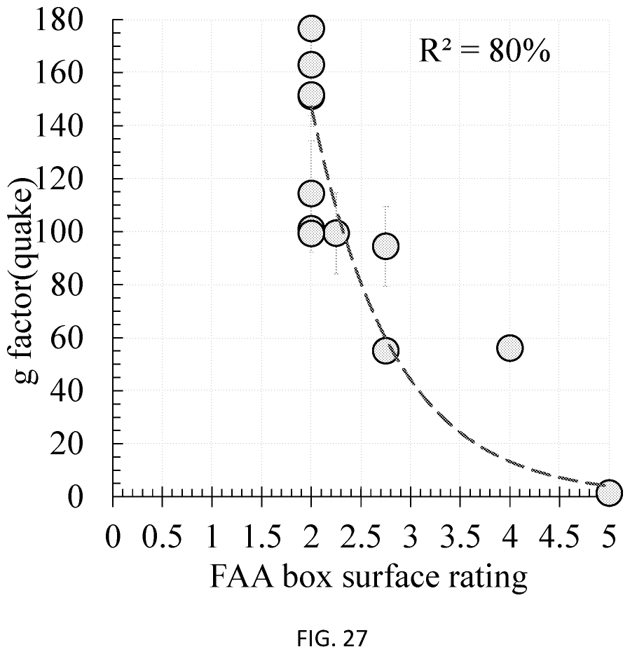

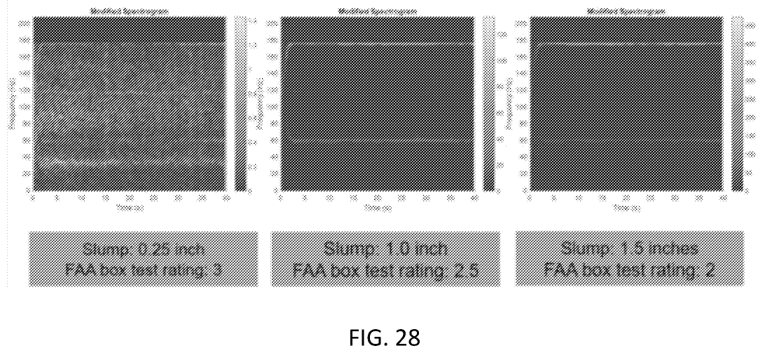

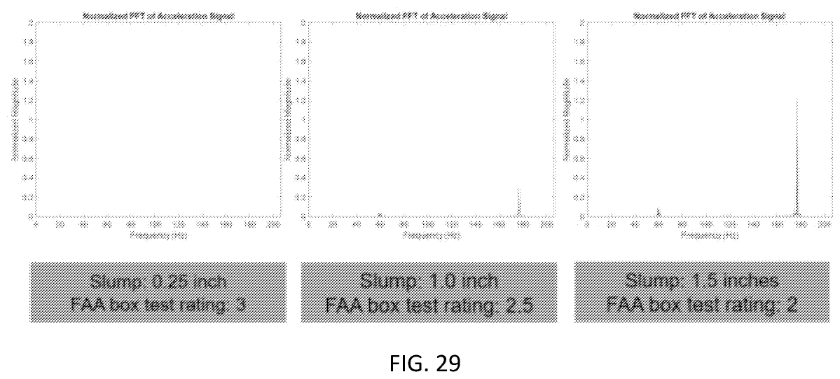

The test may be performed for any duration of time. Optionally, the test may be performed for 40 seconds. The results of such a test are shown in FIGS. 20-29. The method for comparison may be demonstrated using three example mixture performances shown in FIG. 20. The three mixtures shown are merely for demonstrative purposes. Different mixtures and number of mixtures may be used. The comparison may be substantiated with known tests such as the standard ASTM Slump Test and the standard FAA Box Test. For comparison purposes, the results of the Slump Test and Box Test of the three example mixtures shown in FIG. 20 are shown in FIGS. 21 and 22, respectively. FIGS. 23-25 show the resulting magnitudes of transferred energy of the three mixtures using acceleration measurement methods and calculations according to the vibration tests disclosed herein and the resulting g-factors calculated and determined as disclosed herein. Similarly, FIG. 27 plots the g-factor from eleven example mixtures shown in FIG. 26 on the Y axis and Box Test ratings on the X axis. The results illustrate that g-factor may be used as an indicator of workability of fresh concrete. While g-factor may be used as an indicator of workability of fresh concrete, the profile of the magnitude over time plot may be used to analyze the results of mixes having comparable g-factors and different Box Test ratings. For example, a mixture that has a magnitude which increases rapidly over the first few seconds of time may have a better Box Test rating, and therefore better workability than mixtures that have a consistently low magnitude over the first few seconds of time. As shown in FIGS. 28 and 29 the spectrogram and FTT may be further indicators of consolidation and workability of fresh concrete.

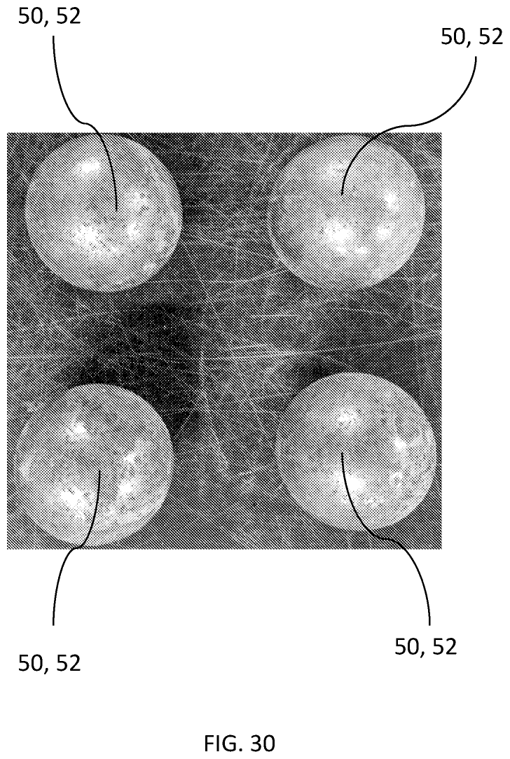

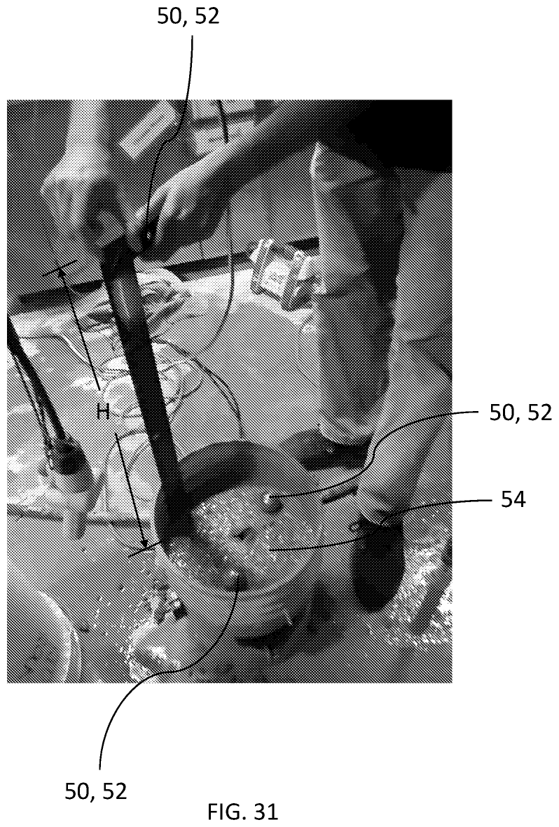

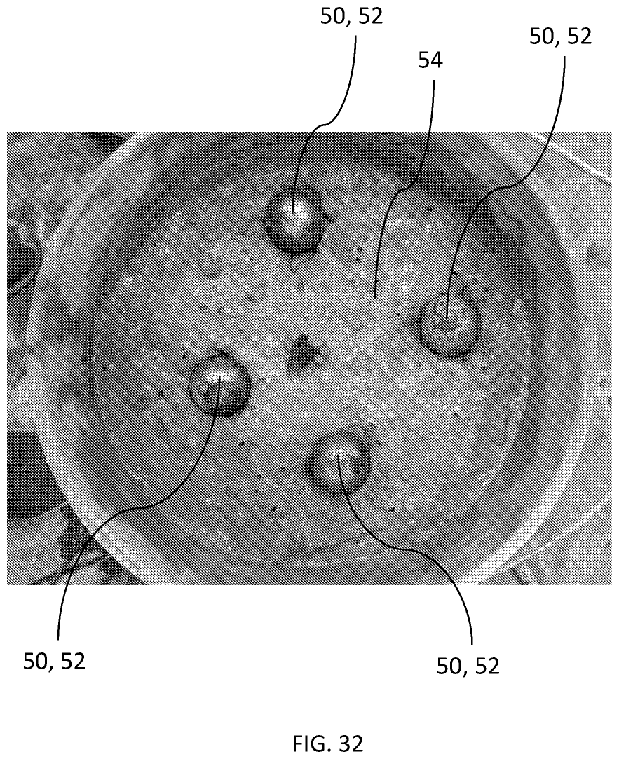

FIG. 30-33 show devices and methods to measure the thickness of the mortar layer, i.e. paste and sand, on the top surface of the consolidated concrete. Optionally, the method may be called the drop test. Experts in airfield runway construction and representatives from the Federal Aviation Agency have expressed concerns that a thicker mortar layer may negatively impact the long-term performance of airfield pavement. As shown in FIG. 30, the method may be performed with at least one weighted object 50, optionally four weighted objects 50. As shown in FIG. 31, the weighted object 50 may be a ball 52. Optionally, the ball 52 may be a steel ball. The ball 52 may weigh about 264 μm and have a diameter of about 1.5 inches.

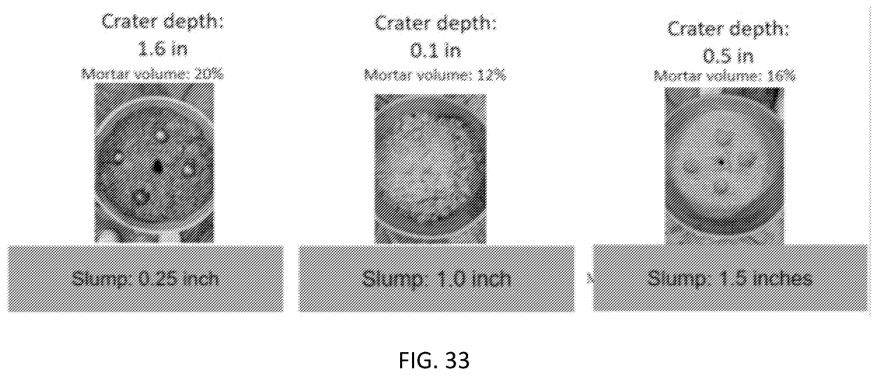

With reference to FIGS. 32 and 33, in an example embodiment, the method comprises:
dropping a first weighted object from a height above the consolidated concrete surface;
removing the first weighted object from the surface of the consolidated concrete surface; and
measuring the depth of the impression left in the consolidated concrete surface from the first weighted object.

Optionally, the method may comprise dropping a second weighted object 50 above the consolidated concrete surface 54 at a different location than the first weighted object, removing the second weighted objection from the surface of the consolidated concrete surface, and measuring the depth of the impression left in the consolidated concrete surface by the second weighted object. The method may be further be repeated with additional weighted objects at different locations, and optionally, two additional weighted objects. Optionally, the weighted objects may be dropped from a height H of 2 feet above the consolidated concrete surface. The depth of the impressions may be measured with a ruler. If more than one weighted object is used to perform the method, the average and standard deviation of all resulting impression depths may be calculated and reported. The results of such a test are shown in FIG. 33. As shown in FIG. 33, which compares the resulting impression or crater depth with the mortar volume, the impression depth may be utilized to measure the mortar thickness of the consolidated concrete.

It is expected that the apparatus and method disclosed and claimed herein will improve the process of laying any concrete but with special focus on very low workability concrete that may be used for slip form concrete applications such as barriers, grain elevators, and roads.

All of the embodiments of the claimed invention described herein are provided expressly by way of example only. Innumerable variations and modifications may be made to the example embodiments described herein without departing from the concept of this disclosure. Additionally, the scope of this disclosure is intended to encompass any and all modifications and combinations of all elements, features, and aspects described in the specification and claims, and shown in the drawings. Any and all such modifications and combinations are intended to be within the scope of this disclosure.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used herein.

Aspect 1: An apparatus for measuring workability of concrete, the apparatus comprising:
a container adapted to receive a sample of the concrete;
a vibrator adapted to be positioned within the container;
a first vibration sensor mounted on the container, the first vibration sensor adapted to generate signals indicative of magnitude and frequency of vibration transmitted through the concrete.

Aspect 2: The apparatus according to aspect 1, wherein the vibrator is adapted to be positioned within the container after the sample is received within the container.

Aspect 3: The apparatus according to aspect 1, further comprising a second vibration sensor mounted on the container.

Aspect 4: The apparatus according to aspect 3, wherein the second vibration sensor is mounted on an opposite side of the container from the first vibration sensor.

Aspect 5: The apparatus according to aspect 1, wherein the first vibration sensor is positioned on an outer surface of the container.

Aspect 6: The apparatus according to aspect 1, wherein the first vibration sensor is positioned to measure the magnitude and frequency of vibration at a point on the container half way between a free surface of the sample and a bottom of the container.

Aspect 7: The apparatus according to aspect 1, wherein the vibrator is adapted to be positioned within a geometric center of the sample.

Aspect 8: The apparatus according to aspect 1, wherein the container is cylindrical.

Aspect 9: The apparatus according to aspect 8, wherein the container has an internal volume of five gallons.

Aspect 10: The apparatus according to aspect 9, wherein the container is formed of a plastic resin.

Aspect 11: The apparatus according to aspect 10, wherein the first and a second vibration sensors are mounted diametrically opposite to one another on an outer surface of the container.

Aspect 12: The apparatus according to aspect 11, wherein the first and second vibration sensors are positioned to measure the magnitude of vibration at a point on the container half way between a free surface of the sample and a bottom of the container.

Aspect 13: The apparatus according to aspect 1, further comprising:

a data collection and display device for receiving the signals from the first vibration sensor, the data collection and display device comprising a visual display for displaying the magnitude of vibration as a function of time.

Aspect 14: The apparatus according to aspect 16, wherein the data collection and display device comprises a computer having resident software to collect and store data, perform various mathematical analyses and drive the display.

Aspect 15: The apparatus according to aspect 16, wherein the data collection and display device comprises one or more electronic filters for conditioning the signals.

Aspect 16: The apparatus according to aspect 1, wherein the vibration sensors comprise accelerometers.

Aspect 17: The apparatus according to aspect 16, wherein the accelerometers have a sampling rate that is configured to measure all or any component of vibrational energy applied to the concrete.

Aspect 18: A method of measuring workability of fresh concrete using the apparatus according to any one of the preceding aspects, the method comprising:
positioning a sample of the fresh concrete within the container;
applying vibration within the sample;
measuring magnitude and frequency of vibration transmitted through the sample to the container;
evaluating the magnitude and frequency of vibration to determine the workability of the fresh concrete.

Aspect 19: The method according to aspect 18, wherein the applying vibration comprises using the vibrator in the sample.

Aspect 20: The method according to aspect 19, wherein the vibration is applied at a geometric center of the sample.

Aspect 21: The method according to aspect 18, wherein the measuring the magnitude comprises using the first vibration sensor.

Aspect 22: The method according to aspect 21, further comprising using the second vibration sensor to measure the magnitude.

Aspect 23: The method according to aspect 22, further comprising using the second vibration sensor to measure the magnitude and frequency at a point on the container opposite to the first vibration sensor.

Aspect 24: The method according to aspect 21, further comprising measuring the magnitude at a point on the container half way between a free surface of the sample and a bottom of the container.

Aspect 25: The method according to aspect 18, further comprising displaying the magnitude of the vibration as a function of time.

Aspect 26: The method according to aspect 25, further comprising calculating an area under a resulting curve of the displayed magnitude of the vibration as a function of time to determine a g-factor; and evaluating the g-factor to determine the workability of the fresh concrete

Aspect 27: The method according to aspect 18, further comprising measuring an acceleration of vibration transmitted through the sample to the container.

Aspect 28: The method according to aspect 27, further comprising applying a low pass filter to the acceleration data to remove frequency above a specified threshold.

Aspect 29: The method according to aspect 28, further comprising applying a total mean removal to the acceleration data to remove drift.

Aspect 30: The method according to aspect 27, further comprising displaying the acceleration data as a function of time.

Aspect 31: The method according to aspect 30, further comprising displaying velocity obtained by integrating the acceleration data as a function of time.

Aspect 32: The method according to aspect 31, further comprising displaying displacement obtained by integrating the velocity data as a function of time.

Aspect 33: A method for comparing a consistency between a first batch of concrete and a second batch of concrete using the apparatus according to any one of aspects 1-17, the method comprising:
positioning a first sample of the first batch within the container;
applying vibration within the first sample;
measuring a magnitude of vibration transmitted through the first sample to the container;
positioning a second sample of the second batch within the container;
applying vibration within the second sample;
measuring a magnitude of vibration transmitted through the second sample to the container;
comparing the magnitude of vibration transmitted through the first sample to the container with the magnitude of vibration transmitted through the second sample to the container.

Aspect 34: The method according to aspect 33, wherein the applying vibration comprises positioning the vibrator into the first and second sample within the container.

Aspect 35: The method according to aspect 34, wherein the vibrator is located at a geometric center of the first and second samples.

Aspect 36: The method according to aspect 33, wherein the measuring the magnitudes of the first and second samples comprises using the first vibration sensor.

Aspect 37: The method according to aspect 36, further comprising using the second vibration sensor to measure the magnitudes of the first and second samples.

Aspect 38: The method according to aspect 37, further comprising using the second vibration sensor to measure the magnitudes of the first and second samples at a point on the container opposite to the first vibration sensor.

Aspect 39: The method according to aspect 36, further comprising measuring the magnitudes of the first and second samples at a point on the container half way between a free surface of the sample and a bottom of the container.

Aspect 40: The method according to aspect 33, further comprising displaying the magnitudes of the first and second samples as a function of time.

Aspect 41: The method according to aspect 40, further comprising calculating an area under a resulting curve of the displayed magnitude of the first sample as a function of time to determine a first g-factor; calculating an area under a resulting curve of the displayed magnitude of the second sample as a function of time to determine a second g-factor; and comparing the first g-factor with the second g-factor.

Aspect 42: A method for determining a thickness of mortar layer of consolidated concrete, the method comprising:
dropping a first weighted object from a height above the consolidated concrete surface;
removing the first weighted object from the surface of the consolidated concrete surface; and
measuring a depth of an impression left in the surface of the consolidated concrete surface from the first weighted object.

Aspect 43: The method according to aspect 42, wherein the weighted object is a steel ball.

Aspect 44: The method according to aspect 42, wherein the method is repeated with a second weighted object.

Aspect 45: The method of aspect 44, wherein the depths of the impressions left in the surface of the consolidated concrete surface from the first weighted object and the second weight object are averaged.

Other embodiments of the present disclosure will be apparent to one skilled in the art. As such, the foregoing description merely enables and describes the general uses and methods of the present disclosure. Accordingly, the following claims define the true scope of the present disclosure.

What is claimed is:

1. An apparatus for measuring workability of concrete, said apparatus comprising:
a container adapted to receive a sample of said concrete;
a vibrator adapted to be positioned within said container;
a first vibration sensor mounted on said container, said first vibration sensor being positioned on an outer surface of said container, said first vibration sensor adapted to generate signals indicative of magnitude and frequency of vibration transmitted through said concrete.

2. (canceled)

3. The apparatus according to claim 1, further comprising a second vibration sensor mounted on said container, wherein said second vibration sensor is mounted on an opposite side of said container from said first vibration sensor.

4. (canceled)

5. (canceled)

6. The apparatus according to claim 1, wherein said first vibration sensor is positioned to measure said magnitude and frequency of vibration at a point on said container half way between a free surface of said sample and a bottom of said container.

7. The apparatus according to claim 1, wherein said vibrator is adapted to be positioned within a geometric center of said sample.

8. The apparatus according to claim 1, wherein said container is cylindrical and is formed of a plastic resin.

9. (canceled)

10. (canceled)

11. The apparatus according to claim 103, wherein said first and a second vibration sensors are mounted diametrically opposite to one another on an outer surface of said container.

12. The apparatus according to claim 11, wherein said first and second vibration sensors are positioned to measure said magnitude of vibration at a point on said container half way between a free surface of said sample and a bottom of said container.

13. The apparatus according to claim 1, further comprising:
a data collection and display device for receiving said signals from said first vibration sensor, said data collection and display device comprising a visual display for displaying said magnitude of vibration as a function of time.

14. (canceled)

15. (canceled)

16. The apparatus according to claim 1, wherein said vibration sensors comprise accelerometers.

17. (canceled)

18. A method of measuring workability of fresh concrete, said method comprising:
positioning a sample of said fresh concrete within said a container;
applying vibration within said sample;
measuring magnitude and frequency of vibration transmitted through said sample to said container;
evaluating said magnitude and frequency of vibration to determine said workability of said fresh concrete.

19. The method according to claim 18, wherein said applying vibration comprises using a vibrator in said sample at a geometric center of said sample.

20. (canceled)

21. The method according to claim 18, wherein said measuring said magnitude comprises using a first vibration sensor.

22. The method according to claim 21, further comprising using a second vibration sensor to measure said magnitude.

23. The method according to claim 22, further comprising using said second vibration sensor to measure said magnitude and frequency at a point on said container opposite to said first vibration sensor.

24. The method according to claim 21, further comprising measuring said magnitude at a point on said container half way between a free surface of said sample and a bottom of said container.

25. The method according to claim 18, further comprising displaying said magnitude of said vibration as a function of time.

26. The method according to claim 25, further comprising calculating an area under a resulting curve of said displayed magnitude of said vibration as a function of time to determine a g-factor; and evaluating said g-factor to determine said workability of said fresh concrete.

27. The method according to claim 18, further comprising measuring an acceleration of vibration transmitted through said sample to said container.

28. (canceled)

29. (canceled)

30. The method according to claim 27, further comprising displaying at least one of said acceleration data, velocity, or displacement as a function of time, wherein said velocity is obtained by integrating the acceleration data as a function of time, and wherein said displacement is obtained by integrating the velocity data as a function of time.

31. (canceled)

32. (canceled)

33. A method for comparing a consistency between a first batch of concrete and a second batch of concrete, said method comprising:
positioning a first sample of said first batch within a first container;
applying vibration within said first sample;
measuring a magnitude of vibration transmitted through said first sample to said first container;
positioning a second sample of said second batch within a second container;
applying vibration within said second sample;
measuring a magnitude of vibration transmitted through said second sample to said second container;
comparing said magnitude of vibration transmitted through said first sample to said second container with said magnitude of vibration transmitted through said second sample to said second container.

34. (canceled)

35. (canceled)

36. (canceled)

37. (canceled)

38. (canceled)

39. (canceled)

40. The method according to claim 33, further comprising displaying said magnitudes of said first and second samples as a function of time.

41. The method according to claim 40, further comprising calculating an area under a resulting curve of said displayed magnitude of said first sample as a function of time to determine a first g-factor; calculating an area under a resulting curve of said displayed magnitude of said second sample as a function of time to determine a second g-factor; and comparing said first g-factor with said second g-factor.

42. (canceled)

43. (canceled)

44. (canceled)

45. (canceled)

46. The method according to claim 33, wherein said first container and said second container are substantially identical or said first container and said second container are the same container.
